(19) United States
(12) Patent Application Publication
(10) Pub. No.: US 2013/0211542 A1
(43) Pub. Date: Aug. 15, 2013

(54) SYNTHESIS OF NANOTOPOGRAPHIC BIOMIMETIC MEMBRANES FOR TISSUE CULTURE, ENGINEERING AND PROSTHETICS APPLICATIONS

(71) Applicant: The Regents of the University of California, (US)

(72) Inventors: Clayton McKee, Davis, CA (US); Christopher J. Murphy, Davis, CA (US); Paul Russell, Davis, CA (US); Shaun P. Garland, Davis, CA (US)

(73) Assignee: The Regents of the University of California, Oakland, CA (US)

(21) Appl. No.: 13/762,290

(22) Filed: Feb. 7, 2013

Related U.S. Application Data
(60) Provisional application No. 61/596,680, filed on Feb. 8, 2012.
(60) Provisional application No. 61/617,281, filed on Mar. 29, 2012.

(51) Int. Cl. A61F 2/02; B29C 59/02; B29C 35/08; B05D 3/10

(52) U.S. Cl. CPC A61F 2/02; B05D 3/107; B05D 3/108; B29C 59/026; B29C 35/0805
USPC 623/23.72; 427/2.1; 216/37; 264/220; 264/494

(57) ABSTRACT

The present invention provide methods for preparing nanostructured membranes. The methods include: providing a substrate with a charged silanized surface; forming a multilayered membrane containing at least two polyelectrolytes; inducing polyelectrolyte phase separation; crosslinking the multilayered membrane; and covalently linking the multilayered membrane to the silanized surface. Methods for fabricating membrane replicas are also disclosed, as well as devices such as cell- and tissue-culture substrates that contain the membranes and membrane replicas. Resulting materials exhibit topographic features and compliance of the extracellular matrix in vivo.

### CROSS-REFERENCES TO RELATED APPLICATIONS

The present application claims priority to U.S. Provisional Patent Application No. 61/596,680, filed Feb. 8, 2012, and U.S. Provisional Patent Application No. 61/617,281, filed Mar. 29, 2012, the entirety of which are incorporated herein by reference.

### STATEMENT AS TO RIGHTS TO INVENTIONS MADE UNDER FEDERALLY SPONSORED RESEARCH AND DEVELOPMENT

This invention was made with Government support under Grant No. RC2AR058971, awarded by the National Institutes of Health. The Government has certain rights in this invention.

### REFERENCE TO A “SEQUENCE LISTING,” A TABLE, OR A COMPUTER PROGRAM LISTING APPENDIX SUBMITTED ON A COMPACT DISK

Not Applicable

### BACKGROUND OF THE INVENTION

It is well established that the physical attributes of the extracellular environment (e.g. topography and/or compliance) can inform internal cell processes and responses to external stimuli. The response of cells to these attributes is particularly relevant given the complex milieu of physical cues that cells can interact with in vivo (often termed biophysical cueing). Biophysical cues are not static and can alter with age, during disease progression, or in response to therapeutic intervention, which has motivated the creation of novel biomimetic cell culture ware in order to obtain a more complete understanding of cell responses to biophysical cues. Biomimetic surfaces, which can be fabricated with natural or synthetic polymers, are typically designed to understand a single physical property of the extracellular environment in isolation.

For example, soft lithography has been used to generate highly ordered topographic arrays of three of the most commonly observed, and studied, aspects of extracellular matrix: ridges and grooves, bumps and pores, all with dimensions in the biological range (nm to μm) (Teixeira AI, et al. J Cell Sci. 2003, 116: 1881-1892; Ghassemi S, et al. J Vac Sci Technol B. 2008, 26(6): 2549-2553; Karuri NW, et al. IEEE Trans Nanobioscience. 2006, 5(4): 273-280). However, the basement membrane of many tissues, including vascular tissues, displays a complex mixture of these topographic features in a stochastic setting. Meanwhile, conventional cell culture ware and other commonly used biomedical devices comprise materials with essentially foreign surface properties, presenting cells and tissues with cues not found in the body and forcing unnatural cellular behavior. Accordingly, there exists an unmet need for device materials that more closely replicate the characteristics of basement cell membranes and other biological surfaces. The methods and materials disclosed herein address this and other needs.

### BRIEF SUMMARY OF THE INVENTION

In one aspect, the present invention provides a method for preparing a nanostructured membrane. The method includes:
a) providing a substrate with a charged silanized surface;
b) forming a multilayered membrane comprising a first polyelectrolyte and a second polyelectrolyte, wherein the membrane contacts the silanized surface;
c) inducing polyelectrolyte phase separation;
d) crosslinking the multilayered membrane; and
e) covalently linking the multilayered membrane to the silanized surface.

In some embodiments, the method also includes:
f) preparing a composite stamp using the crosslinked multilayered membrane as a template;
g) imprinting an uncured polymer substrate with the stamp; and
h) curing the polymer substrate to form a membrane replica.

In a second aspect, the invention provides a method for preparing a compliant nanostructured surface. The method includes:
a) contacting a surface with (3-acryloxypropyl)-trichlorosilane;
b) forming an aqueous film between the surface and a nanostructured mold, wherein the film comprises polyethylene glycol diacrylate and a photoinitiator, to form an imprinted uncured polymer substrate; and
c) exposing the imprinted uncured polymer substrate to ultraviolet light to form the compliant surface.

In a third aspect, the invention provides a nanostructured membrane including:
a) a substrate with a silanized surface; and
b) a crosslinked multilayered membrane contacting the silanized surface and comprising a first polyelectrolyte and a second polyelectrolyte;
   wherein the crosslinked membrane is covalently linked to the silanized surface, and wherein the crosslinked membrane comprises pores ranging from about 50 nanometers to 1.5 micrometers.

In a fourth aspect, the invention provides polymer-based nanostructured membrane replicas.

In a fifth aspect, the invention provides cell culture substrates and implantable prosthetic devices that include the novel membranes and/or membrane replicas described herein.

### BRIEF DESCRIPTION OF THE DRAWINGS

FIG. 1a shows an atomic force microscope height mode image of the NOA81 replica of the porous polyelectrolyte membrane (scale bar 2 μm).

FIG. 1b shows a scanning electron microscope image of inferior vena cava basement membrane from a Rhesus macaque (scale 600 nm) (Liliensiek, et al. Tissue Eng Part A. 2009, 15(9): 2643-2651; reprinted with permission from Mary Anne Liebert, Inc. Publishers).

FIG. 2a shows the hypothetical cell migration path from an initial time point, Tᵢ, to a final time point T_{f}.

FIG. 2b demonstrates measurement of the cell turning angle as a function of the cell path, as well as for increasing time interval, τ.

FIG. 3 shows the migration rate of both HUVECs and HAECs was significantly increased when cultured on the synthetic membrane as compared to the flat surfaces (top panel). Proliferation rates were significantly decreased for both cell types, when cultured on the synthetic membrane (***≡p<0.001, **≡p=0.01) (bottom panel).

FIG. 4 shows the directional persistence of HUVECs and HAECs as a function of substrate type.

FIG. 5 shows the gene expression of MMP-2, SPARC, and PECAM in HUVECs cultured on flat or synthetic membrane surfaces. Expression of MMP-2 and SPARC decreased when cells were cultured on the topographic membrane (p<0.001 for MMP-2). PECAM expression was unaffected by the topography.

FIG. 6 shows the gene expression for MMP-2, SPARC, PECAM, and ICAM-1 in HAECS, both in the presence and absence of 10 ng/ml TNFα, as a function of substrate topography. Significance values for various comparisons are located in the boxes below the bar graphs.

FIG. 7 shows a schematic description of the formation of a thin-film topographic hydrogel. A thin film of prepolymer solution is created between a PDMS mold and glass surface that has been silanated with 3-APT. After exposure to high intensity UV light, the PDMS mold is removed. In solution, the expansion of the crosslinked hydrogel is constrained in the plane of the glass slide due to the covalent bonds formed between the film and glass slide, as well as by the thickness of the film.

FIG. 8 shows AFM images of a 4000 nm-pitch ridge-and-groove surface and fluorescent images of corneal epithelial cells adhered to the hydrogel surface.

FIG. 9 shows the molded ridge-and-groove topographies in the thin film hydrogels, ranging from 2000-800 nm pitch.

FIG. 10 shows AFM images of simulated basement membrane. The scale bar in the AFM images is 1 micrometer.

FIG. 11 shows AFM images of a PEG-based laponite hydrogel surface characterized by reversible nanostructured topography.

### DETAILED DESCRIPTION OF THE INVENTION

### I. General

The present invention provides nanostructured membrane materials that present stochastic topographic cues that closely mimic the topographic environment of many vascular basement membranes found in the body. As such, the materials provide a means for controlling a number of important endothelial cell behaviors. The ease of fabrication and the ability to replicate the porosity of the nanostructured membranes using soft lithography makes these substrates an ideal construct to investigate topographic cueing on cellular behavior. The versatility of the methods also allows for production of materials with an elastic modulus similar to tissues in vivo. The materials can be used as substrates for tissue engineering and cell culture. Furthermore, incorporation of these materials onto bioengineered surfaces can facilitate long-term acceptance of vascular implants, given the significantly decreased expression of several cardiovascular disease-related genes observed for cultured cells grown in response to TNFα on these substrates.

### II. Definitions

As used herein, the term “nanostructure” refers to a plurality of physical features in a material, wherein the features have any dimension (e.g. height, width, and length) under 1.5 microns. Typically, nanostructure features are characterized by two or more dimensions under 1 micron. Such features include ridges, grooves, bumps, and pores.

As used herein, the term “membrane” refers to a material spanning a substrate and containing one or more polyelectrolyte layers. A “multilayered” membrane refers to a material containing one or more layers of a first electrolyte and the same number of layers of a second polyelectrolyte. The first and second polyelectrolytes have opposite charges, and the layers of the first polyelectrolyte contact the layers of the second polyelectrolyte in an alternating fashion.

As used herein, the term “polyelectrolyte” refers to a polymer having a plurality of ionic functional groups. A polyelectrolyte can be a cationic polyelectrolyte, wherein the functional groups are entirely or predominantly positively charged, or an anionic polyelectrolyte, wherein the functional groups are entirely or predominantly negatively charged.

As used herein, the term “phase separation” refers to the self-segregation of two or more immiscible polymers in a mixture so as to minimize unfavorable interactions between the immiscible polymers. Phase separation can give rise to polymer features ranging in size from a few nanometers to several microns. The term “inducing polyelectrolyte phase separation” refers to causing unfavorable interactions between polyelectrolytes that would otherwise interact favorably, such that phase separation occurs. Inducing polyelectrolyte phase separation can include neutralizing ionic functional groups. Inducing polyelectrolyte phase separation can include exposing a membrane to a solution having an acidic pH or a basic pH, or to a solution containing ions that pair with a polyelectrolyte functional group via electrostatic interactions.

As used herein, the term “substrate” refers to a surface that can support a membrane. Substrates can be made from plastics, glass, or other materials and can include articles such as microscope slides, tissue culture ware, and silicon wafers. Substrates may be cleaned, chemically modified, or otherwise manipulated prior to forming membranes.

As used herein, the term “silanized” refers to a surface that has been modified via covalent bond formation with a silane reagent. The silane reagents are typically alkoxysilanes that can form covalent bonds with hydroxyl groups on the surface of a substrate. Examples of suitable silanes include, but are not limited to [3-(2-aminoethylamino)propyl]trimethoxysilane, (3-aminopropyl)trimethoxysilane, and N¹-(3-trimethoxysilylpropyl)diethylenetriamine.

As used herein the term “crosslinking” refers to forming covalent bonds between one or more components in a nanostructured membrane. A polyelectrolyte chain can be crosslinked to one or more additional polyelectrolyte chains, for example, or to functional groups on a silanized surface. Crosslinking can also occur between two functional groups on the same polyelectrolyte chain. Crosslinking can be conducted by contacting the membrane with a chemical crosslinker, or by exposing the membrane to light or elevated temperatures.

As used herein, the term “stamp” refers to a device containing an elastomer-based block with a structured face characterized by nanoscale topography. The structured face is used to imprint the topographical features into a suitable polymer substrate. A “composite stamp” refers to a stamp containing two or more elastomer-based blocks in contact with each other.

As used herein, the term “polymer substrate” refers to a polymer surface that is sufficiently pliable for imprinting of a topographical pattern by a stamp. An “uncured polymer substrate” refers to a pliable polymer that becomes rigid upon treatment with chemical reagents, heat, or light, a process referred to herein as “curing.”

As used herein, the term “imprinting” refers to transferring a topographic pattern from a stamp to a pliable polymer substrate by pressing the stamp into the substrate.

As used herein, the term “membrane replica” refers to a cured polymer substrate characterized by a nanostructured topography that is similar or substantially identical to a nanostructured membrane “master”. The membrane replica is produced by imprinting the topographic features on an uncured polymer substrate with a stamp fabricated using the nanostructured membrane master.

As used herein, the terms “cell culture substrate” and “tissue culture substrate” refer to a surface that can support in vitro subsistence and/or proliferation of cells and tissues. A tissue culture substrate can be used in a vessel such as a well-plate or a culture flask. A tissue culture substrate can contain a nanostructured membrane or membrane replica fabricated via the methods of the present invention.

As used herein, the term “implantable prosthetic device” refers to an article intended to replace a naturally occurring organ or tissue within a subject. In certain cases, the prosthetic device can promote attachment and proliferation of cells or tissues in vivo. A prosthetic device can be a vascular implant such as a stent. A prosthetic device can contain a nanostructured membrane or membrane replica fabricated via the methods of the present invention.

### III. Embodiments of the Invention

The methods disclosed herein provide access to biomimetic membranes using inexpensive and cell-friendly materials. Furthermore, the use of soft lithography allows for the replication of membrane topography in devices such as cell culture ware. Such materials can be used to elucidate how the combination of multiple topographic features influences cell behavior.

Accordingly, the present invention provides a method for preparing a nanostructured membrane. The method includes:
a) providing a substrate with a charged silanized surface;
b) forming a multilayered membrane comprising a first polyelectrolyte and a second polyelectrolyte, wherein the membrane contacts the silanized surface;
c) inducing polyelectrolyte phase separation;
d) crosslinking the multilayered membrane; and
e) covalently linking the multilayered membrane to the silanized surface.

Any suitable substrate may be used in the methods of the present invention. For example, the substrate can be selected from commercially available culture ware including well plates, cell culture dishes, tissue culture flasks, Petri dishes, and the like. The substrate can be a microscope slide. In some embodiments, the substrate may be plastic or glass. Examples of plastics for use as substrates include, but are not limited to, polystyrene, polyethylene, and poly(methylmethacrylate). Silicon wafers are also contemplated for use as substrates in the methods of the present invention. The substrate can be cleaned or otherwise treated prior to fabrication of the nanostructured membranes. For example, the substrate may be exposed to ultraviolet light or ionized plasma, such as an oxygen plasma, prior to membrane fabrication. Alternatively, the substrate can be treated with a solution of acid or base and an optional detergent prior to membrane fabrication. Treatment prior to membrane fabrication can be sufficient to change the surface properties of the substrate such as the hydrophilicity or wettability.

The substrate can be chemically modified to anchor polyelectrolytes or otherwise stabilize the nanostructure membranes. In some embodiments, the substrate is silanized by covalently modifying the substrate surface with a silane. Suitable silanes for substrate modification include, but are not limited to, allyltriethoxysilane, [3-(2-aminoethylamino)propyl]trimethoxysilane, (3-aminopropyl)trimethoxysilane, (3-acryloxypropyl)-trichlorosilane, diethoxy(3-glycidyloxypropyl)methylsilane, 3-(triethoxysilyl)propyl isocyanate, 3-(trimethoxysilyl)propyl acrylate, and N¹-(3-trimethoxysilylpropyl)diethylenetriamine. Modification is conducted under conditions sufficient to form covalent bonds between the substrate surface and the silane. The substrate surface can be contacted, for example, with a solution of a silane in water or another suitable solvent. Alternatively, the substrate surface can be contacted with silane vapor. The substrates can be contacted with silane vapor under ambient conditions or under reduced pressure using a vacuum. Unreacted silane can be removed from the substrate by storing the substrate under a vacuum. In some embodiments, modification of a substrate with a silane results in a charged silanized surface. The surface charges can promote anchoring of polyelectrolytes via electrostatic interactions. In some embodiments the invention provides methods for preparing nanostructured membranes as described above, wherein providing a substrate with a charged silanized surface includes contacting the substrate with (3-aminopropyl)trimethoxy silane (APS).

In general, the multilayered membranes employed in the methods of the present invention are assembled via layer-by-layer deposition of oppositely charged polyelectrolytes in an alternating fashion. The multilayer membrane is stabilized by electrostatic interactions between a first polyelectrolyte (a negatively-charged anionic polyelectrolyte or a positively-charged cationic polyelectrolyte) and a second polyelectrolyte (having the opposite charge). The material properties of the multilayered membranes depend in part on the number of polyelectrolyte layers in the membrane. For example, the number of layers can effect membrane durability, membrane transparency, and other important characteristics. For certain applications, including light microscopy of cells and tissues, membrane transparency is a particularly desirable characteristic. As such, any suitable number of polyelectrolyte layers may be assembled in the methods of the present invention. In some embodiments, the methods of the invention include forming a multilayered membrane containing 5-25 layers of the first polyelectrolyte and an equal number of layers the second polyelectrolyte, wherein the layers of the first polyelectrolyte and the second polyelectrolyte contact each other in an alternating fashion. The multilayered membrane can contain, for example, 5-20, 5-15, 5-10, 10-25, 10-20, 10-15, 15-25, or 15-20 layers of the first polyelectrolyte and an equal number of layers of the second polyelectrolyte. In some embodiments, the multilayered membrane includes 10-15 layers of the first polyelectrolyte and an equal number of layers of the second polyelectrolyte. Forming a multilayered membrane can also include assembling one or more additional polyelectrolyte layers on top of the multilayered membrane. For example, an additional layer of the first polyelectrolyte may be assembled on top of the multilayered membrane.

Any suitable polyelectrolytes may be used in the methods of the present invention. Examples of suitable negatively-charged anionic polyelectrolytes include, but are not limited to, poly(acrylic acid), poly(styrene sulfonate), poly(vinyl sulfate), poly(2-acrylamido-2-methyl-1-propanesulfonic acid), polyanetholesulfonic acid, poly(methacrylic acid). Examples of suitable positively-charged cationic polyelectrolytes include, but are not limited to, poly(allylamine hydrochloride), poly(L-lysine), poly(vinylpyridine), poly(diallyldimethylammonium chloride), polyethyleneimine, and poly(N-vinylamine). One of skill in the art will recognize that the molecular weight of the polyelectrolytes can contribute to the physical properties of the multilayered membrane, such as membrane thickness and durability as well as nanostructure morphology upon polyelectrolyte phase separation. As such, polyelectrolytes with any molecular weight suitable to achieve a desired membrane property may be used in the methods of the present invention. In some embodiments, the first polyelectrolyte is an anionic polyelectrolyte. In some embodiments, the first polyelectrolyte is a cationic polyelectrolyte. In any case, the first polyelectrolyte and the second polyelectrolyte have opposite charges. In some embodiments, the first polyelectrolyte is poly(acrylic acid). In some embodiments, the second polyelectrolyte is poly(allylamine hydrochloride).

The multilayer membranes of the present invention are assembled by coating the substrate with a first layer of the first polyelectrolyte and then coating the first layer with a layer of the second polyelectrolyte. Assembly continues by forming additional layers of the first polyelectrolyte and the second polyelectrolyte in an alternating fashion. The layers can be formed by dipping or otherwise immersing the substrate in polyelectrolyte solutions. Any suitable immersion time may be used for layer formation. For example, the substrate may be immersed in a polyelectrolyte solution for anywhere between 1 minute and 1 hour. In some embodiments, the substrate is immersed in a polyelectrolyte solution for 15 minutes. Washing via immersion in water or another solvent can be conducted between formation of different polyelectrolyte layers. Alternatively, layers can be formed by spin-coating the substrate with the polyelectrolyte solutions. In some embodiments, the solutions are aqueous solutions. The pH of the solutions can be adjusted by adding an acid such as hydrochloric acid or a base such as sodium hydroxide. In general, the pH of a solution is adjusted to ensure partial or complete ionization of the polyelectrolyte. The polyelectrolyte solutions can optionally contain salts, including but not limited to sodium chloride and magnesium chloride, to modulate the electrostatic interactions between polyelectrolyte layers. One of skill in the art will recognize that membrane properties such a thickness and durability will depend in part on solution pH and the presence of optional salt additives, as well as the polyelectrolyte concentration in the solutions. In some embodiments, a 10 mM solution of poly(acrylic acid) with a pH around 4.0 is used for forming the multilayered membrane. In some embodiments, a 10 mM solution of poly(allylamine hydrochloride) with a pH around 7.5 is used for forming the multilayered membrane. After membrane formation, excess solvent can be removed by drying the membrane in an oven under ambient pressure or reduced pressure. In some embodiments, the membrane is dehydrated by drying in an oven for 1 hr at 60-80° C.

Nanostructured topography results from polyelectrolyte phase separation in the multilayered membrane. In some embodiments the present invention provides methods for preparation of nanostructured membranes as described above, wherein inducing polyelectrolyte phase separation includes contacting the multilayered membrane with an acidic aqueous solution. In some embodiments, the multilayered membrane is immerse in an aqueous solution of ˜5 mM hydrochloric acid for one minute. Other acid concentrations and immersion times are also contemplated for use in the methods of the present invention. Surprisingly, membrane nanostructure was found to vary significantly with slight variations in acid dipping concentrations, allowing for fine tuning of the membrane topography. In some embodiments, the membrane is dehydrated by drying in an oven after phase separation. The resulting grooves, ridges, and pores mimic the topography of the vascular basement membrane and other extracellular surfaces.

The methods of the present invention include stabilizing the nanostructured membranes after polyelectrolyte phase separation by crosslinking the multilayered membrane and covalently linking it to the silanized surface of the substrate. These steps may be conducted sequentially or concurrently. Crosslinking the multilayered membrane may be conducted using a chemical crosslinker. Chemical crosslinkers may be heterobifunctional crosslinkers, which react with different functional groups, or homobifunctional crosslinkers, which react with like functional groups. Chemical crosslinkers may also be non-specific crosslinkers, which react with membrane components regardless of specific functional groups. Examples of suitable chemical crosslinkers include, but are not limited to, dialdehydes, diamines, succinimidyl esters, imidoesters, and arylazides. Covalently linking the multilayered membrane to the silanized surface of the substrate can also be conducted using the chemical crosslinkers described above. In some embodiments the present invention provides methods for preparation of nanostructured membranes as described above, wherein crosslinking the multilayered membrane and covalently linking the membrane to the silanized surface of the substrate are performed concurrently by contacting the multilayered membrane and the substrate with a chemical crosslinker. In some embodiments, the chemical crosslinker is glutaraldehyde. In some embodiments, the multilayered membrane is immersed in a solution of 2.5% glutaraldehyde after polyelectrolyte phase separation. Alternatively, the linking steps described above can be conducted by heating the multilayered membrane after polyelectrolyte phase separation. In the case of membranes containing poly(allylamine hydrochloride) and poly(acrylic acid), for example, heating the membranes promotes amide bond formation between the acrylic acid groups and the allylamine and surface-bound propylamine groups. In some embodiments the present invention provides methods for preparation of nanostructured membranes as described above, wherein crosslinking the multilayered membrane and covalently linking the membrane to the silanized surface of the substrate are performed concurrently by heating the multilayered membrane and the substrate. In some embodiments, crosslinking the membrane and anchoring the membrane to the substrate is conducted by heating the phase-separated membrane at 200° C. for 20 minutes. Membrane stabilization after polyelectrolyte phase separation ensures that the nanotopography is preserved and the membrane remains anchored on the substrate for periods of several days or longer during cell culture or tissue culture. This stabilization can prevent topography degradation and substrate detachment that characterizes certain materials lacking either membrane crosslinking or the silanized substrate surface for membrane anchoring.

Although the sensitive nature of the phase separation process provides an opportunity to fine-tune membrane nanotopography as described above, it also leads to a certain degree of inherent variability in the materials produced by the inventive methods. This variability is undesirable in scenarios where multiple experiments are required, such as behavior comparisons of different tissues on a given substrate. Surprisingly, the nanostructured membranes proved sufficiently robust to serve as “masters” for the formation of elastomer-based stamps that can be used to reproducibly transfer membrane nanotopography to useful substrate materials via soft lithography. Accordingly, some embodiments of the present invention provide a method for preparation of a nanostructured membrane as described above, wherein the method also includes:
f) preparing a composite stamp using the crosslinked multilayered membrane as a template;
g) imprinting an uncured polymer substrate with the stamp; and
h) curing the polymer substrate to form a membrane replica.

In general, the stamps used for the soft lithography process are composed of elastomers such as poly(dimethylsiloxane) (PDMS). The stamp composition can vary, but should allow for maintenance of the membrane topography during stamp fabrication and topography transfer with high resolution during stamping. In particular, a “clean” transfer will depend on mechanical properties such as stamp hardness and stiffness, as well as the size range of the topographical features. As described herein, a stamp with a composite architecture preserves the topography of the nanostructured membrane and cleanly transfers the topographic pattern to polymer substrates. The composite stamp contains a thin layer of hard PDMS including the topographic pattern and a thicker support layer of flexible PDMS. The stamps can be fabricated with commercially available elastomer products. A layer of surfactant, such as sodium dodecyl sulfate and the like, coating the nanostructured membrane allowed the stamp to be cleanly lifted from the “master” before use. Accordingly, the present invention provides a method for the preparation of a nanostructured membrane as described above, wherein preparing a composite stamp includes:
i) coating the crosslinked multilayered membrane with a surfactant;
ii) forming a thin layer of stiff poly(dimethylsiloxane) (PDMS) in contact with the surfactant-coated membrane;
iii) forming a thick layer of flexible PDMS in contact with the thin layer of stiff PDMS; and
iv) removing the resulting two-layer composite stamp from the multilayered membrane.

The nanostructured stamps can be used to transfer membrane topography to a number of useful polymer substrates. In general, the stamp is used to imprint an uncured polymer substrate that is sufficiently pliable to conform to the topography on the structured face of the stamp. The pliable polymer substrate can be a thermosetting polymer that hardens upon heat-curing, such as certain epoxy resins and polyurethanes. Alternatively, the flexible polymer substrate can be a UV-curable polymer that hardens upon exposure to ultraviolet light, including several “light cure adhesives” that are known in the art. The UV-curable polymer can be a mercaptoester-type polymer, also known as a thiol-ene polymer. Examples of UV-curable mercaptoesters are described in U.S. Pat. Nos. 3,661,744; 4,157,241; and 5,028,661. Suitable UV-curable mercaptoesters include, but are not limited to, NOA 71, NOA 81, and NOA 83H, available from Norland Products, as well as UV15-7, available from Master Bond. Other polymers can also be used in the methods of the invention. Various articles, such as microscope slides or cell culture well plates, can be coated with an uncured polymer or pre-polymer, imprinted with a stamp, and cured to provide topographically defined membrane replicas with high reproducibility. In some embodiments, the present invention provides methods as described above, wherein imprinting an uncured polymer substrate with a composite stamp includes:
ii) coating a surface with a pre-polymer comprising a mercapto-ester; and
ii) pressing the composite stamp into the pre-polymer to form an imprinted uncured polymer substrate.

In some embodiments, imprinted uncured polymer substrates comprising mercapto-esters are cured by exposing the uncured polymer to UV light.

Hydrogels can also be used as a polymer substrate for imprinting with nanoscale features using composite stamps. Hydrogels are an attractive way to recreate the stiffness of soft tissues in cell culture due to the ease in which the stiffness of the gel can be modified, as well as being compatible with many cell types. Any suitable hydrogel can be employed in the methods of the present invention. Examples of hydrogels include, but are not limited to, naturally occurring polymers such as agarose, methylcellulose, and hyaluronan; polyacrylamides; polyvinyl alcohols; polyvinylpyrrolidones; polyethylene glycols; and silicone hydrogels. Stiffness of certain hydrogels can be modified by crosslinking polymer chains in a hydrogel. The degree of crosslinking can correlate with the stiffness of the hydrogel. In some embodiments of the present invention, the hydrogel used as a substrate for imprinting with composite stamps contains crosslinked polyethylene glycol (PEG). Surfaces for cell culture or other applications can be functionalized with crosslinked PEG by polymerizing reactive PEG-based monomers, such as polyethylene glycol diacrylate (PEGDA), in the presence of a photoinitiator such as 4-(2-hydroxyethoxy)phenyl-(2-hydroxy-2-propyl)ketone (commericially available as Irgacure 2959) and a suitably reactive surface. Reactive surfaces are prepared, for example, by contacting plastic or glass substrates with silane reagents including, but not limited to, (3-acryloxypropyl)-trichlorosilane. When a polymerization mixture containing PEGDA and a photoinitiator is allowed to react on an acryloxy-functionalized surface, the resulting crosslinked PEG hydrogel is covalently anchored to the surface. The hydrogel can be “imprinted” with nanotopographic features by sandwiching the polymerization mixture as a film layer between the functionalized surface and a composite stamp as described above. The PEGDA may be of any molecular weight suitable for forming compliant hydrogels.

Accordingly, some embodiments of the present invention provide methods as described above, wherein imprinting an uncured polymer substrate with a composite stamp includes:
i) contacting a surface with (3-acryloxypropyl)-trichlorosilane; and
ii) forming an aqueous film between the surface and the composite stamp, wherein the film comprises polyethylene glycol diacrylate and a photoinitiator, to form an imprinted uncured polymer substrate.

The drawback of very soft hydrogels, in respect to topographic cueing, is their large swelling ratios. This material property makes it difficult to create a molded or photo-induced topographic feature that will maintain its shape in solution, as they are not rigid enough to maintain their shape. Surprisingly, sufficiently thin films were found to hold the submicron topographic features of the composite stamps when swollen in solution. Without wishing to be bound by any particular theory, it is believed that lateral expansion of the hydrogel is mechanically constrained in the plane of the surface while the hydrogel is free to expand vertically. By selecting PEG derivatives with the appropriate molecular weight and choosing appropriate polymerization conditions, topographic surfaces with compliance properties similar to many soft biological tissues (elastic moduli ˜100 kPa) can be formed.

One of skill in the art will appreciate that the technique can be use to fabricate surfaces characterized by any number of patterns. Any pattern that can be imprinted using a suitable mold, such as a PDMS stamp, is intended for use in the methods of the present invention. Accordingly, some embodiments of the invention provide a method for preparing a compliant, nanostructured surface. The method includes:
a) contacting a surface with (3-acryloxypropyl)-trichlorosilane;
b) forming an aqueous film between the surface and a nanostructured mold, wherein the film comprises polyethylene glycol diacrylate and a photoinitiator, to form an imprinted uncured polymer substrate; and
c) exposing the imprinted uncured polymer substrate to ultraviolet light to form the compliant surface.

In some embodiments, the nanostructured mold is a PDMS stamp.

The compliant surfaces of the present invention are ideal for the study of cell behavior, as PEG hydrogels are non-toxic, fouling resistant, and can be modified by a large number of small molecules, including saccharides, peptides, DNA, inorganic materials, and proteins to target specific cell-substrate interactions. In some embodiments, substances such as collagen, fibronectin, laminin, polylysine, or an RGD peptide (that is, a peptide containing an arginine-glycine-aspartic acid sequence) can be incorporated into the hydrogels to promote cellular adhesion or other cellular behaviors.

In some embodiments, inorganic materials can be incorporated into the hydrogels so as to provide surfaces with “switchable” nanotopographic features. In some embodiments, the hydrogel is formed by polymerizing polyethylene glycol diacrylale (PEGDA) in the presence of synthetic laponite clay. Addition of the laponite modifies the material properties of the hydrogel thin films, such that the films can reversibly switch between a flat surface and a high fidelity nanotopography formed by soft lithography. Without wishing to be bound by any particular theory, it is believed that the laponite modifies the mechanical properties of the PEGDA film, facilitating both the retractability and long-term “memory” of the nanotopography of the thin film hydrogel, by physically crosslinking the PEGDA.

Accordingly, some embodiments of the present invention provide a method for preparing a compliant, nanostructured surface as described above, wherein the aqueous film further comprises laponite particles and the compliant nanostructured surface can be reversibly switched between a flat surface and a surface with nanotopographic features.

The methods of the invention can be used to provide a number of useful materials. In some embodiments, the invention provides a nanostructured membrane. The nanostructured membrane includes:
a) a substrate with a silanized surface; and
b) a crosslinked multilayered membrane contacting the silanized surface and comprising a first polyelectrolyte and a second polyelectrolyte;
   wherein the crosslinked membrane is covalently linked to the silanized surface, and wherein the crosslinked membrane comprises pores ranging from about 50 nanometers to 1.5 micrometers. The silanized surface and multilayered membrane comprise the chemical components described above. The nanostructured membranes can also include one or more additional components selected from peptides, proteins, cell ligands, therapeutic drugs, and nanoparticles. Such components can provide cellular cues in tissue engineering and stem cell engineering applications. Some embodiments of the present invention provide a membrane replica as described above. In some embodiments, compliant nanostructured surfaces as described above are provided.

The nanostructured membranes, membrane replicas, and compliant nanostructured surfaces can be incorporated into other articles such as equipment for cell culture and biomedical implants. Such articles accurately reflect the ubiquitous presence of nanotopographic features found in basement membranes with the body. As such, they are an attractive alternative to traditional culture ware and other biomaterials that present non-natural cues to cells and therefore force unnatural cell behavior. In some embodiments, the invention provides a cell culture substrate comprising a membrane or membrane replica as described above. In some embodiments, the invention provides a tissue culture substrate comprising a membrane or membrane replica as described above. In some embodiments, the invention provides an implantable prosthetic device comprising a membrane or membrane replica as described above. The prosthetic device can be, for example, a vascular implant.

### IV. Examples

The following examples are intended to illustrate, but not to limit, the invention as disclosed above.

### EXAMPLE 1

### Synthesis of Nanostructured Biomimetic Membrane Materials

Nanostructured membrane fabrication. Stabilized nanoporous membranes were generated by forming porous polyelectrolyte membranes (PEMs) of poly(acrylic acid) (PAA, MW˜60,000, Polysciences, Inc., Warrington, Pa.) and poly(allylamine hydrochloride) (PAH, MW˜160,000, Alfa Aesar, Ward Hill, Mass.) on glass microscope slides. Solutions of PAA and PAH were prepared at 0.01 M (based on MW of monomer unit) at a pH of 3.5 and 7.5, respectively. Solutions were prepared using ultra-high purity water (18.2 MΩ.cm) and the pH was controlled with NaOH and HCl. Before PEM formation, the glass microscope slides were plasma treated (Harrick Plasma Cleaner, Hayrick Plasma, Ithaca, N.Y.) for one minute and then immediately silanized by exposure to 3-aminopropyltrimethoxysilane (APS, Sigma Aldrich) vapor for 1 hour under vacuum. After 1 hour in the presence of APS vapor, the APS was removed and the samples were left under vacuum for 24 hours to remove unreacted APS. These amine-terminated glass slides were initially submerged in PAA for 15 minutes, followed by two, one-minute submersions in ultra-high purity water, and then submerged in PAH for 15 minutes. This process was repeated until ten layers (PAA/PAH=one layer) were generated. At the end of the dipping process, the samples were blown dry with high-purity nitrogen and then dehydrated in an oven at 60° C. for 1 hour. Samples were then dipped in a solution of HCl at a pH of 2.3 for 1 minute, which resulted in a highly porous, phase separated PEM. Samples were immediately blown dry with high-purity nitrogen and placed in an oven at 60° C. for 1 hour. If these porous PEMs were re-exposed to solution, the porous structure was rapidly lost. To maintain the long-term stability of the porous structure of the PEM, the APS coated slide containing the PEM was placed in an oven at 200° C. for 20 minutes. This cross-links the PAA and PAH, as well as the base PAA layer to the amine terminated glass slide via amide bond formation. PEMs that were not cross-linked to an APS coated glass slide would typically detach from the surface if left in solution for more than three or four days (PEMs crosslinked to the APS did not detach). Although detachment is useful in recovering the thin film from the glass, cell culture experiments dictate the PEM to remain attached to the glass. Alternatively, the PEMs can be used as “master” templates for producing nanolithography stamps as described below.

Nanolithography. The following procedure was used to recreate identical replicas of the “master” porous PEM described above. The master PEMs were first dipped in a 2×CMC (critical micelle concentration) solution of sodium dodecyl sulfate and then blown dry with ultrahigh purity nitrogen. This step ensured that subsequent PDMS molds of the topography detached, without also damaging the PEM on the glass surface. High-fidelity replicates of the nanoporous features of the PEM were created using a composite two-layer PDMS stamp, consisting of a thin and hard PDMS layer (hPDMS) attached to a thick support of 184 PDMS.

The composite stamp was formed as follows: 2 drops of 2,4,6,8-tetramethyl-2,4,6,8-tetravinylcyclotetrasiloxane (396281-10ML, Sigma Aldrich) and 50 μl of platinum-divinyltetramethyldisiloxane complex in xylene (SIP6831.2-LC, Gelest, Morrisville, Pa.) were gently mixed with 3.4 grams of 7.0-8.0% (vinylmethylsiloxane)-dimethylsiloxane copolymer, trimethylsiloxy terminated (VDT-731, Gelest). This solution was quickly degassed (1-2 min).

One gram of 25-30% (methylhydrosiloxane)-dimethylsiloxane copolymer, trimethylsiloxane terminated (HMS-301, Gelest) was gently mixed with the degassed solution. A dime-sized volume of the polymer solution was poured onto the center of the “master” porous PEM, which was then spun at 4000 rpm for 40 seconds. This thin film was cured for 30 minutes at 60° C. to produce the hPDMS layer. At the end of 30 minutes a thick layer of Sylgard 184 PDMS was poured onto the slide and was cured for 3 hours at 60° C. Once the PDMS was cured, the composite mold was peeled from the “master” PEM. Subsequent replicas of the PEM were generated by molding NOA81 (Norland Products) to the composite PDMS stamp, by first spin coating NOA81 to a plasma cleaned microscope slide at 4000 rpm for 40 seconds and then pressing the PDMS stamp into the thin film. This was subsequently UV-cured for 40 minutes.

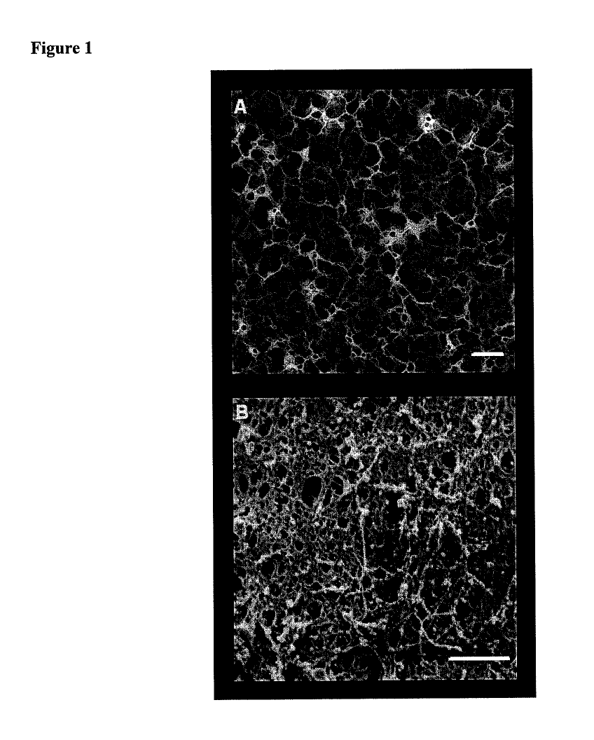

FIG. 1a is an AFM height image of the NOA81 replica of the porous PEM and will be referred to as a synthetic membrane. Finally, flat surfaces of NOA81 were generated via spin coating and UV-curing so that the cellular behaviors on the synthetic membranes could be compared to flat surfaces of identical chemistry.

Atomic Force Microscopy (AFM). The topographic features of the porous surfaces were quantified from the piezo “height” response of a contact mode image obtained with an atomic force microscope (MFP-3D-BIO, Asylum Research, Santa Barbara, Calif.). The surfaces were scanned using silicon nitride cantilevers, which have a square pyramid tip incorporated at the free end (k=0.06 N/m, PNP-TR-50, Nano And More, Lady's Island, S.C.). To ensure an undistorted view of the actual sample, Asylum's Argyle Light software was used to match the Z-scale of the topographic features (their height) to the lateral X and Y dimension of the image (i.e. a 1:1 aspect ratio).

Results and discussion. During the formation step of the porous PEM, the initially transparent PEM-coated slide became slightly cloudy after exposure to the acidic solution and the opacity was proportional to the thickness of the film. For this reason PEMs were limited to ten layers. The opacity of the porous PEM was also transferred to the NOA81 replicas (NOA81 flat surfaces cured as fully transparent solid), indicating the cloudiness was due primarily to light scattering from the outermost face of the topography and not precipitate formation within the PEM film. In solution, the opacity was almost undetectable and had no apparent effect on the clarity of phase contrast images of the cells on the synthetic membrane. PEM surfaces were not used directly for cell culture, as the porous structure was found to be exquisitely sensitive to slight differences in dipping conditions such as pH. Therefore, it was difficult to obtain exact replicates between experiments. (The pH of the dipping solution was found to increase with each PEM coated slide that was dipped, as hydrogen ions were depleted from solution. To avoid this problem, a large volume of HCl solution was prepared from which aliquots for each dipped PEM were removed.) The NOA81 replicas however, could be mass-produced with identical features from a single master PEM that best represented native basement membrane. This was particularly helpful for conducting multiple experiments and directly comparing differences between cell types. FIG. 1a is a 20 μm² AFM height image of the NOA81 replica of the porous polyelectrolyte multilayer. This topography was conserved over multiple imaged positions on the replica. FIG. 1b is an SEM image of inferior vena cava basement membrane from a Rhesus macaque (Liliensiek, et al. Tissue Eng Part A. 2009, 15(9): 2643-2651). The synthetic membrane in FIG. 1a comprised pores, ranging from ˜200 nm up to ˜1.5 μm, with the majority of pores being submicroscopic (pore depth ranged from ˜50 to 300 nm). The surface also comprised ridges and bumps that were interconnected with the porous network, meeting the criteria for a biomimetic topography that comprised the three major topographic components of in vivo basement membrane.

A number of aspects associated with these highly porous substrates make them ideal candidates for studying the relationship between cellular behavior and topographic cues. First and foremost is the similarity of the synthetic membrane to the basement membrane of in vivo tissues. These substrates intrinsically possess a number of the common topographic features of basement membranes with stochastic surface order. They are easy to fabricate, with the porosity of the substrate easily modified by slight changes in the pH of the dipping solution. In contrast to another PEM-based cell study (Hajicharalambous, et al. Biomaterials. 2009, 30(23-24): 4029-4036), the present study employs soft lithography to fabricate a large number of these surfaces with identical topographies. This process helps to decrease the systematic error associated with reproducibility of the porous features on separate surfaces. Soft lithography also allows for replicates of the PEM topography to be generated on any piece of cell culture ware, as well as for the potential to transfer the topography onto implantable bioengineered surfaces. However, if the chemistry of the PEM is experimentally required, amine-termination of the glass slide with APS will ensure that the porous PEM remains bound to the glass during long-term cell culture conditions. By using a small number of PEM layers, these porous substrates were also sufficiently transparent and could be used in a number of light microscopy related cell and molecular biology experiments. Finally, as mentioned previously, the topographic features associated with vascular basement membranes are conserved in a number of other in vivo basement membranes. This makes these synthetic topographic substrates highly relevant for diverse fields of investigation.

### EXAMPLE 2

### Study of Cell Proliferation and Migration on Nanostructured Biomimetic Membranes

Cell culture. Human umbilical vein endothelial cells (HUVECs) and human aortic endothelial cells (HAECs) were maintained in endothelial basal media supplemented with EGM-2 BulletKit (Lonza, Walkerville, Md.). The BulletKit contains GA-1000, hEGF, fetal bovine serum, heparin, ascorbic acid, R3-IGF, VEGF, hFGF-B and hydrocortisone. During culture and migration assays, cells were maintained at 37° C. and 5% CO₂.

Cell Migration Analysis via a Custom Automated Cell Tracker. Time-lapse observations of cells were obtained on both the flat and membrane surfaces over a 12-hour time period, with a collection rate of one frame every eight minutes (90 frames). Cells were imaged in phase contrast using an inverted light microscope (Zeiss, Axio Observer Al, Carl Zeiss, Thornwood, N.Y.) with a 10× objective. Cells from a minimum of ten separate locations were imaged for each condition. Cells were plated at 75,000 cells per plate, which ensured a cell population that was sparse enough for single cell tracking. Cell inclusion for analysis was limited to those cells that remained within the frame during the entire 12-hour time-lapse and to those that did not undergo division. This resulted in cell counts of 30 to 50 cells for each condition.

Manual cell tracking (visual location and recording of the cell X and Y pixel data) is a very time consuming and subjective process. Therefore, an algorithm was written that automatically tracks and records the location of a cell as its position changes during the course of the time-lapse movie. The source code for this algorithm was written with the programming and statistical analysis software package IgorPro (Wavemetics, Lake Oswego, Oreg.). The algorithm works by tracking a user specified dark or bright location within the cell. The migration path of all cells in this study were collected by tracking the darkest region of the cell, which in phase contrast microscopy at 10× magnification, is predominantly the central region of the cell. The “manual tracking” feature available in Axiovision 4.6 (Carl Zeiss, Germany) was used as a controlled test to compare the output of the new custom algorithm. The user interface for the new automated cell tracker allows users to modify a small number of tracking parameters to ensure a successful track, as well as the option of “manual tracking” if the automated tracker is unsuccessful. Statistical significance and p-values for the migration assays were determined by t-test analysis (mean±standard deviation).

The automated tracker was found to successfully follow the position of a moving cell over the data collection time interval. The darkest location within the cell was tracked for all cell tracks. With a 10× objective, proper Köhler illumination, and tracking parameters, the algorithm-chosen tracked position was centrally located within the cell. This position was often observed to be nucleoli within the nucleus. The automated tracker successfully tracked upwards of 80% of the cells that met the inclusion criteria for a given cell population, negating the need for manual tracking. Given the X and Y pixel coordinates of the moving cell and the scale of the image (micrometers/pixel), it was easy to simultaneously output the distance moved and migration rate. The efficiency of the automated tracker was also quantified by comparing the time required to automatically track the location of the 40 HUVEC cells with the time required to manually track the cells using Axiovision. The automated tracker typically tracked a cell in less than 10 seconds and required approximately 30 minutes to track and output the results (migration, proliferation, and persistence) for all the included cells within the ten positions collected for analysis. Manually tracking the same cells using the Axiovision software took three times longer than automated tracking (including analysis of migration rate). The influence of the substratum topography on the trend in migration rate of HUVEC cells on the synthetic membrane and flats were consistent between the automated tracker and Axiovision's manual tracker.

Cell proliferation. Five-day proliferation assays were conducted to measure the proliferation rate of the endothelial cells and were performed in triplicate. Cells were plated at 100,000/plate on day 1 and then imaged the following day to determine the initial cell count. The cell count of the slide was determined by collecting five images along the length of the slide, determining the average number of cells/image and then using this to determine the approximate number of cells per plate. Cells were then left in an incubator at 37° C. and 5% CO2 until day five. On day five, the plates were re-imaged and the approximate number of cells per plate was again determined. The proliferation rate was quantified by determining the ratio of the number of cells on day 5 to the number of cells on day 2. To compare the difference between cells cultured on the flat surfaces to cells cultured on the synthetic membrane, the results were normalized to the proliferation rate measured on flats (i.e. normalized proliferation rate of one for flats). Statistical significance and p-values for the proliferation assays were determined by t-test analysis (mean±standard deviation).

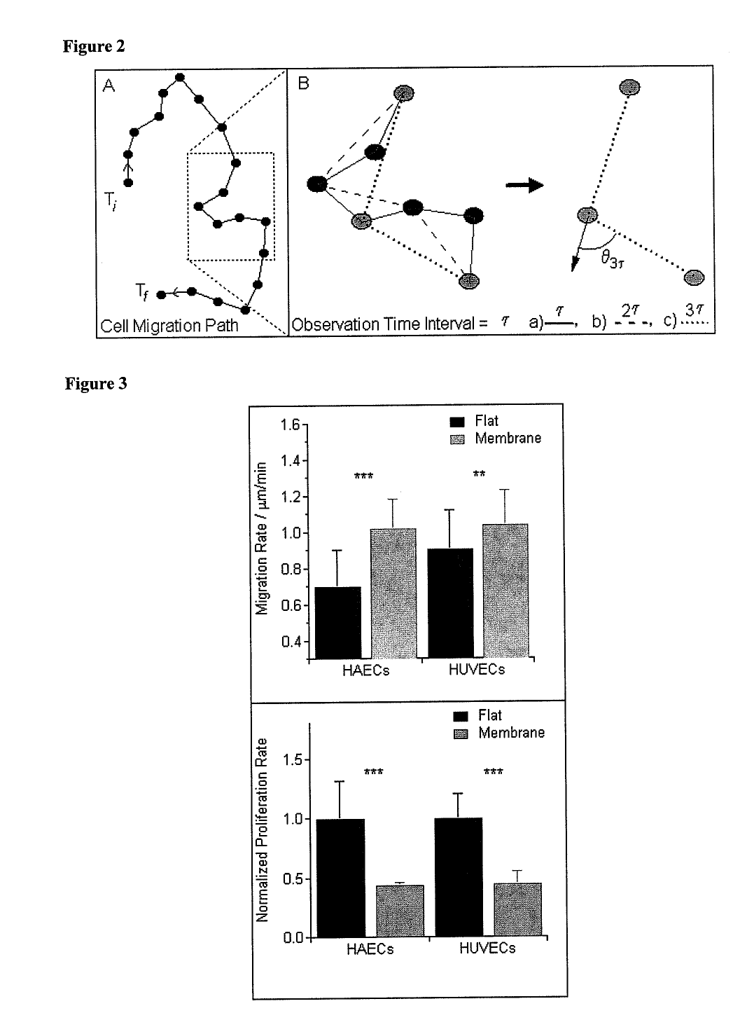

Cell directional persistence. Cells in motion tend to maintain their direction of motion for small periods of time. This is known as directional persistence. To determine the influence of the synthetic membrane on directional persistence of the cells, histograms of the observed turning angle between three separate positions in the migration path of a cell were generated as a function of increasing time interval between observation positions. This is demonstrated graphically in FIG. 2. FIG. 2b, in particular, demonstrates how the cell turning angle is measured as a function of the cell path, as well as for increasing time interval, τ. Three different observational time intervals are demonstrated (a-solid line, b-dashed line, and c-dotted line). The turning angle θ is defined by the angle formed between a forward progression of the intermediate time point and the final time point (demonstrated for observation time interval of 3 τ). The shorter the time interval, the larger the number of measured turning angles. For example, the number of measured turning angles in the subset path in (B) would be five for τ, three for 2τ and one for 3 τ. With increasing time interval, the measured cell turning angle is increasingly distributed in a random fashion between 0 and 180°.

From compiled values of measured turning angles, histograms were generated for the frequency of 0-10° turns (persistent motion) and 170-180° turns (anti-persistent) as a function of increasing observation time interval. For small time intervals, a histogram of turning angles should show an increased number of persistent turns as compared to anti-persistent. As the time interval increases, the frequency of persistent turns decrease and the anti-persistent turns increase. At large enough time intervals, cells would appear to have random motion and a histogram of turning angles would be evenly distributed between 0-180°. The time interval of persistent motion is defined by visually noting the point in which the frequency of persistent turns becomes constant with increasing time interval. If the measured turning angles beyond this time interval are random, the frequency of persistent and anti-persistent turns should also coincide with one another.

Results. Both HUVECs and HAECs (as well as corneal epithelial and human trabecular meshwork cells, data not shown) adhere, spread, migrate and proliferate on the topographic substrates, without the need for additional chemical coatings, such as fibronectin or collagen, to assist in cell adhesion. Cells in culture remained viable through confluence, indicating no deleterious effects from the topography or leaching of chemicals from the UV-cured NOA81. The migration and proliferation rate of these cells on the synthetic membrane and flat surfaces are demonstrated in FIG. 3. Both HUVECs and HAECs had similar migration rates on the synthetic membrane surfaces, with values of 1.04±0.16 and 1.02±0.19 μm/min respectively (FIG. 3, top panel). The absence of the substratum topography on migration rate was however, more pronounced for HAECs. The effect of the synthetic membrane was to increase the rate of both cell types (on flats, HUVECs—0.91±0.21 and HAECs—0.70±0.20 μm/min). The trend between the migration rate for HUVEC cells on the synthetic membrane and flats was consistent when manually tracked with Axiovision 3.6. However, when manually tracked, the absolute magnitudes in migration rates were increased for both substrate conditions (1.03±0.12 and 0.84±0.22 μm/min, membrane and flat respectively). The proliferation rate was significantly reduced for both cell types with normalized proliferation rates that were approximately half the rate for cells cultured on flat surfaces (FIG. 3, bottom panel). Non-normalized results demonstrated that HUVECs proliferated approximately 1.4 times faster than HAECs.

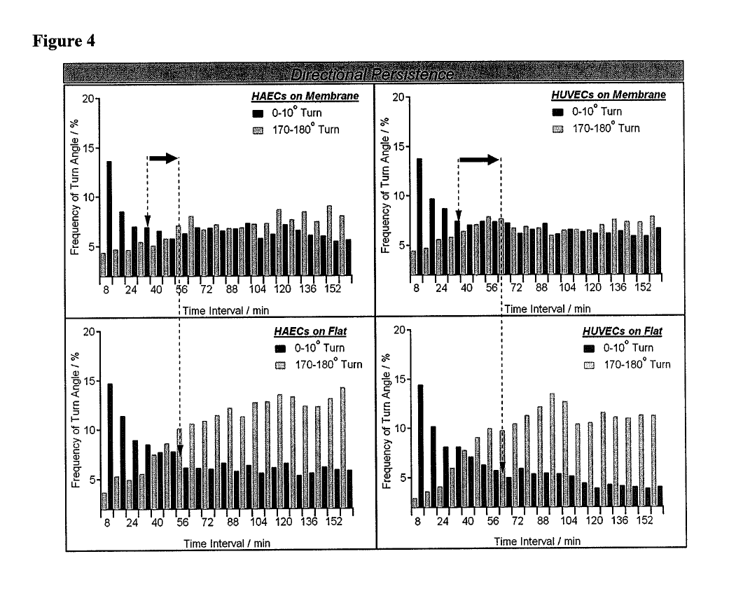

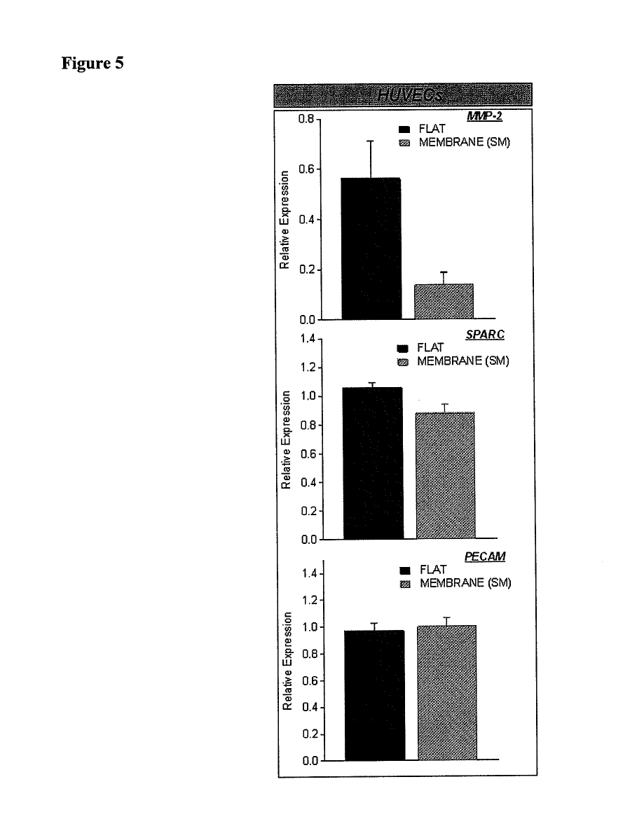

FIG. 4 shows how the synthetic membrane influences the directional persistence of migrating cells for both substrate conditions. As the observation time interval increased, the frequency of measured persistent turns (0-10° turns) decreased as expected. Both cell types demonstrated reduced directional persistence time on the synthetic membrane compared to flats. The time interval of directional persistence was approximately 30 minutes for both HAECs and HUVECs on the synthetic membrane and approximately 60 minutes on the flat surfaces. The loss of directional persistence over these time frames was also correlated with an increase in anti-persistent migration (170-180°). With increasing time interval, beyond the time frame of persistent motion, both cell types on the flat surfaces had an increased probability of returning on the same path they just migrated upon. This is demonstrated by an increasing percentage of anti-persistent turns over persistent turns after the time interval of directional persistence. Both cell types did not preferentially migrate either forward or backwards along their migration path on the membrane surface after the loss in directional persistence, as noted by the similarity in the percentage of persistent and anti-persistent turns with increasing time interval.

Discussion. In aggregate, these topographically biomimetic surfaces had a significant impact on a number of cell behaviors involved in both the homeostasis and degradation of vascular tissues. The motility of both HUVECs and HAECs was significantly altered by the presentation of the biomimetic topographies, both in terms of the velocity at which the cells migrate, but also in terms of the cells' ability to maintain their directional persistence during migration. Migration and directional persistence are important factors in such biological processes as embryogenesis, immune response and wound healing. In the absence of externally directed cell migration, e.g., durotaxis or chemotaxis, both the HUVECs and HAECs in this study were observed to have an intrinsic directional persistence time that was influenced by the stochastic topography of the underlying surface. Changes in directional persistence time, associated with the static stiffness (not durotaxis) of the topography, may shed light on the important role that stochastic topographies play on the residence time of cells in tissues whose mechanics have been altered by disease, therapeutic intervention, or by the wound healing process. Patterned, non-random ridges and grooves on biomimetic length scales have previously been shown to guide both HUVEC and HAEC migration along the path of the ridges (Liliensiek, et al. Biomaterials. 2010, 31(20): 5418-5426). In the case of HUVECs, both migration and proliferation rate decreased significantly. In the present case, these endothelial cells are interacting with an assortment of ridges, pores and bumps that are randomly presented to the adhered cell. Consequently, the cells are not contact guided by long-range order of the substratum and therefore have a decreased directional persistence time. In contrast to the patterned ridge and groove topographies, migration rates were increased on these substrates for both cell types.

On biomimetic topographic features, both HUVECs and HAECs were shown here to have a decreased proliferation rate as compared to flat surfaces. A link between nuclear volume and proliferation rate has recently been suggested for cells on the ridge and groove topographies as well as for other topographies. Cytoskeletal and nuclear shape changes associated with culture on the synthetic membranes may impact the proliferation rate of the HUVECs and HAECs.

### EXAMPLE 3

### Analysis of Gene Expression in Endothelial Cells Cultured on Biomimetic Nanostructured Membranes

Gene expression assays. Both HUVECs and HAECs were plated at 100,000 cells per plate and allowed to culture for three days on the flat and membrane surfaces prior to RNA extraction. Expression of SPARC (secreted protein acidic and rich in cysteine), PECAM (platelet endothelial cell adhesion molecule), and MMP-2 (matrix metalloproteinase-2) was quantified for both HUVECs and HAECs. The influence of TNFα (tumor necrosis factor-alpha) on the expression of ICAM-1(intercellular adhesion molecule-1) in HAECs was also studied. For those experiments, HAECs were exposed to 10 ng/ml TNFα for 12 hours prior to RNA extraction. RNA was extracted using the Qiagen RNeasy kit according to the manufacturer's protocol (Qiagen, Valencia, Calif.). Semi-quantitative real-time PCR was performed using 75 ng of RNA per reaction and a one-step TaqMan kit with commercially available aptamers for SPARC, PECAM, MMP-2 and ICAM-1 (Applied Biosystems, Calif.). Reverse transcription occurred for 30 minutes at 50° C. followed by 95° C. for 10 minutes. Amplification of the cDNA occurred for forty cycles at 60° C. for 1 minute, followed by 95° C. for 15 seconds. GAPDH (glyceraldehydes 3-phosphate dehydrogenase) served as the internal control gene. At least three reactions were run for each sample and the experiment was performed in triplicate. Gene expression was normalized to the expression of mRNA from cells on flat surfaces. Statistical significance and p-values for gene expression results were determined using Sigma Plot 11 (Sustat Software, Chicago, Ill.) with the following tests: For HUVECs and HAECs, Mann-Whitney Exact Rank Sum Tests were used to determine significance between substrates. For HAECs, Kruskal-Wallis Exact One Way Analysis of Variance on Ranks tests were used to determine significance among groups. Significance was set at p<0.05 for all analyses (mean±standard error of the mean).

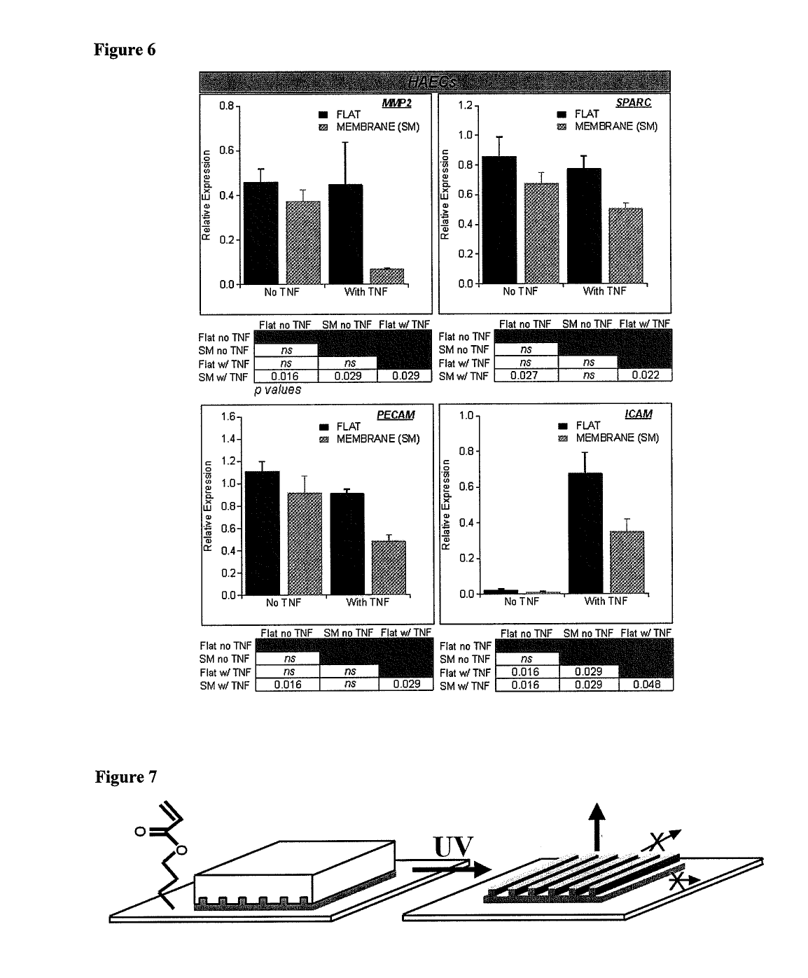

Results. The influence of the topographic membrane on gene expression was dependent on cell type, as shown in FIG. 5 and FIG. 6. The bar graphs demonstrate the relative gene expression as function of substrate type. Both MMP-2 and SPARC trended towards decreased expression when HUVECs were cultured on the topographic membrane; the difference in MMP-2 was significant (p<0.001) (FIG. 5). Below the bar graphs in FIG. 6 there are tables that show the p-values when various conditions for HAEC culture are compared. For example, there was a significant decrease (p=0.048) in the expression of ICAM-1 when HAECs cultured on the synthetic membrane were exposed to 10 ng/ml TNFα for 12 hours (SM w/TNF) as compared to the flat surface with TNFα (Flat w/ TNF). The combined influence of the topography and TNFα had a marked effect on the expression MMP-2, SPARC, and PECAM in HAECs as well, with all being significantly down-regulated on the synthetic membrane as compared to flats. With the exception of ICAM-1, cells cultured on flat surfaces had no significant response to TNFα. Although the trends in gene expression of MMP-2 and SPARC were similar for both HUVECs and HAECs, only HUVECs had a significantly down-regulated expression for MMP-2 when cultured on the synthetic topographically patterned membrane. For both cell types, the synthetic topographically patterned membrane had no significant effect on the expression of PECAM, indicating the endothelial cell type was unaltered by the topographic cues.

Discussion. In-vivo regulation (SPARC) and remodeling (MMPs) of extracellular matrix occurs in a number of important biological processes, including angiogenesis, wound healing, invasion and inflammation. MMP-2 was significantly decreased by the presentation of the synthetic membrane in HUVECs, indicating a decreased remodeling response to the surface that best reflected the in-vivo topographic environment of the cell. Although the trends were similar for expression of MMP-2 in HAECs, it was not significantly altered by the presentation of the topography alone. Similar to other reports of endothelial cells on substratum topography, these synthetic membranes did not change PECAM expression, although TNFα is known to down-regulate the expression of PECAM. No previous investigations into the expression of PECAM in HAECs on a biologically relevant topography in the presence of inflammatory cytokines are known to have taken place. All of the genes were significantly decreased (as compared to flats) when cells were exposed to elevated levels TNFα, a known factor associated with end stage heart failure. MMP-2 expression has been shown to be increased and participates in the matrix remodeling that occurs during atherosclerotic lesion formation. The dramatic down regulation of MMP-2 in TNFα-treated HAECs on the topographically patterned membrane suggests the in-vivo topography may influence both the onset and progression of cardiovascular disease. SPARC is known to be important in collagen deposition and remodeling in multiple forms of cardiovascular disease. The down regulation of SPARC in TNFα-stimulated HAECs on the synthetic membrane demonstrates that a biologically relevant topography may be important in modulating the inflammatory response of arterial endothelial cells. The expression of ICAM-1 on the synthetic membrane was also significantly decreased for HAECs, when the cells were induced to express ICAM-1 by exposure to TNFα. In aggregate these result suggests that the inflammatory response of HAECs may be altered by substratum topographic cues alone and indicates the potential to decrease inflammation by decreasing matrix remodeling and the potential for adhesion and invasion of leukocytes on a full endothelial cell layer. Bioengineered implants with similar topographies may therefore have a decreased probability of rejection. The presence of biomimetic cues in the membranes can also participate in stabilizing cellular phenotype and contribute to the maintenance of homeostasis.

### EXAMPLE 4

Experimental Section. Glass microscope slides (Fisher Scientific, 12-544-1) were plasma treated for 1 min prior to vapor phase silanation with 3-acryloxypropyl trichlorosilane (3-APT, Gelest, SIA0199.0) for 2 hrs under vacuum. After silanation, the glass slides remained under vacuum for 24 hrs to remove unreacted 3-APT. PEGDA (Sigma Aldrich, 701963, mw=6,000 gm/mol) solutions were prepared at 20% w/v in a solution containing 0.15% w/v Irgacure 2959 (BASF Chemicals). After overnight equilibration, a three microliter volume of the prepolymer solution was placed on top of the silanated glass slide and then a one square centimeter PMDS mold was gently placed on top, resulting in the a thin capillary film. Fabrication of the PDMS molds was conducted as described above. The samples were then closed in a container of ultrahigh purity nitrogen gas for two hours. The sample and container were placed under high intensity UV for 20 minutes (365 nm, ˜35 mW/cm²). PDMS molds were then gently peeled from the crosslinked thin film. An Asylum Research MFP-3D-BIO was used to image the topographies in contact mode. Silicon nitride cantilevers (k=0.32N/m, PNP-TR-50, Nano And More, Lady's Island, S.C.) were used for imaging. Samples were either imaged immediately after removal of the stamp or were stored in water for imaging at 24 hrs. Imaging with the smallest possible force provided the best image quality in solution.

To promote cell adhesion on the hydrogel surfaces, collagen (Advanced BioMatrix, PureCol®, 5005-B) was added at 5% v/v to the prepolymer solutions. Immortalized human corneal epithelial cells (Robertson, D. M. et al. Invest Ophthalmol Vis Sci 2005, 46, 470) in tissue culture medium were plated on the topographies and incubated for 24 hours at 37° C. and 5% CO₂. Cells were fixed and stained for the actin cytoskeleton and nucleus and then imaged with a Zeiss inverted light microscope (Carl Zeiss, Axiovert 200M) using a 20× objective.

Results and discussion. The stiffness of extracellular matrix has been measured by a number of techniques and often the elastic component of a viscoelastic tissue is quantified and then subsequently used to create compliant cell culture-ware. This can create difficulties, as the range of published elastic modulus (EM) values for a given tissue typically span several orders of magnitude. A recent review on the published values of EM has demonstrated that a major contributor to the variation is a result of the method by which the EM was measured (McKee, C. T., et al., Tissue Eng Part B Rev 2011, 17, 155). Indentation measured values of EM are, on average, consistently smaller than the tensile measurement of EM for the same tissue. The indentation measured elastic modulus of many soft tissues range from 0.1-200 kPa, which may better represent what cells ‘sense’ as the compliance of BM over the much stiffer tensile measurement of EM. Hydrogels are an attractive way to recreate the stiffness of soft tissues in cell culture due to the ease in which the stiffness of the gel can be modified, as well as being compatible with many cell types. The drawback of very soft hydrogels, in respect to topographic cueing, is their large swelling ratios. This material property makes it difficult to create a molded or photo-induced topographic feature that will maintain its shape in solution, as they are not rigid enough to maintain their shape.

FIG. 7 schematically outlines the process employed to address these issues. Glass microscope slides with covalently bound 3-(acryloxypropyl trichlorosilane) were used as the base substrate. A small drop (3 μL) of a prepolymer PEGDA (6000 g/mol)/Irgacure 2959 solution is placed on the glass slide and then a one square centimeter topographic mold (PDMS) is placed on top of the solution. This produces a thin film between the glass slide and mold. The topographic PDMS molds used for this experiment had either a ridge-and-groove pattern having 4000-400 nm pitch (pitch=ridge width+groove width), or the basement membrane-type topography formed from the polyelectrolyte membranes as described here. The PDMS-PEGDA-glass sample was then placed in a closed container of nitrogen gas for two hours to displace excess oxygen in the prepolymer solution, which acts as a radical scavenger and prevents proper cross-linking of the acrylate groups on the PEGDA chain. The molded prepolymer solutions were exposed to high intensity UV light for 20 minutes while still under a nitrogen atmosphere. After cross-linking, the PDMS was gently peeled from the substrate and the samples were either immediately imaged with atomic force microscopy, or placed in ultrahigh purity water for later imaging. No crack propagation or peeling was observed for these very thin films, indicating a strong bond formed between the acrylate groups on the PEGDA backbone and the acrylate groups on the glass surface.

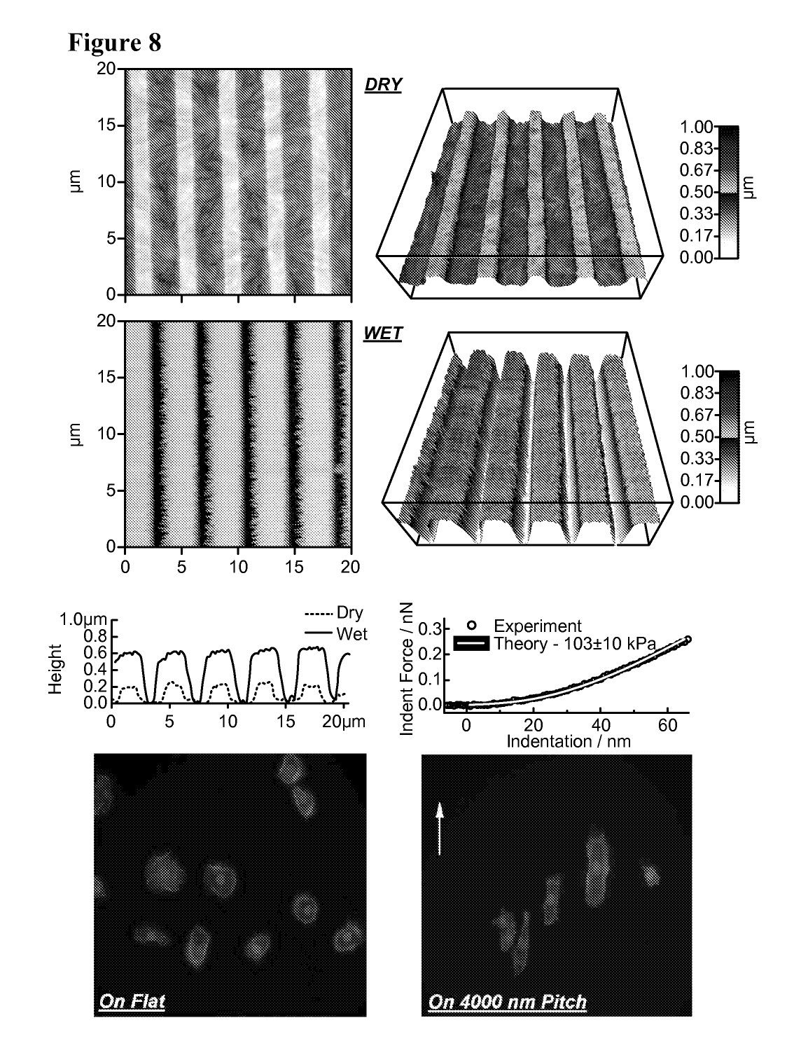

Immediately after the removal of a 4000 nm pitch mold, the cross-linked PEGDA was visually observed to have the correct topography (as noted by the colored diffraction of light from the ridge and grooves). As water in the thin film evaporated, the substrate then rapidly clouded over due to semi-collapse of the ridges. FIG. 8 shows AFM images of a 4000 nm pitch ridge and groove surface and fluorescent images of corneal epithelial cells adhered to the hydrogel surface. The top image is of the dry film post crosslinking. The image was taken approximately 10 minutes after removal of the PDMS mold. The middle image is of the topography after 30 minutes of exposure to ethanol. The elastic modulus (117 kPa) was measured over ridges. The bottom images demonstrate that corneal cells randomly oriented when adhered to flat glass surfaces and when adhered to the 4000 nm pitch PEGDA surface, they align to the long axis of the ridge and groove. Type one collagen was used to induce cell adherence on the hydrogel surfaces (see methods). FIG. 8 (top) is an AFM height image of a dry/collapsed 4000 nm pitch film. In addition to the ridges and grooves, stress lines are observed in the collapsed film. The sample was swollen in ethanol for 30 minutes and then re-imaged at the same location (FIG. 8, middle). Imaging in ethanol minimized adhesions between the AFM tip and soft sample, which could introduce artifacts in the image. Using a lower molecular weight PEGDA (700 g/mol), to decrease tip substrate adhesions and increase film rigidity, resulted in similar adsorption of both ethanol and water by PEGDA.

From the two AFM images, the swelling ratio of the thin film was estimated by comparing the volume of a ridge in the dry state to the same ridge in the wet state. The column volume can be accurately determined from the AFM image dimensions of the ridge. The swelling ratio (Vol_{wet}/Vol_{dry}), as a percentage, for the 6000 mw PEG film was 570%. Force vs. indentation data was well described by assuming a rigid cone (AFM tip) indenting a plane elastic solid (see: A. E. H. Love, Q J Math 1939, 10, 161). FIG. 8 (bottom) depicts human corneal epithelial cells, with fluorescently labeled actin network and nucleus, adhered to a flat glass surface and a 4000 nm-pitch ridge-and-groove PEGDA surface. Type one collagen (5% v/v) was incorporated into the prepolymer solutions, so that cells would be able to adhere to the hydrogel surface. Cells seeded on the 4000 nm-pitch surface responded to the soft ridge and groove topography by aligning with the long axis of the topography (a close inspection of the cells on the right reveal the underlying ridge and groove). The morphological change of the cells on the molded substrate demonstrates the robustness of the topography to be present and active for biological applications.

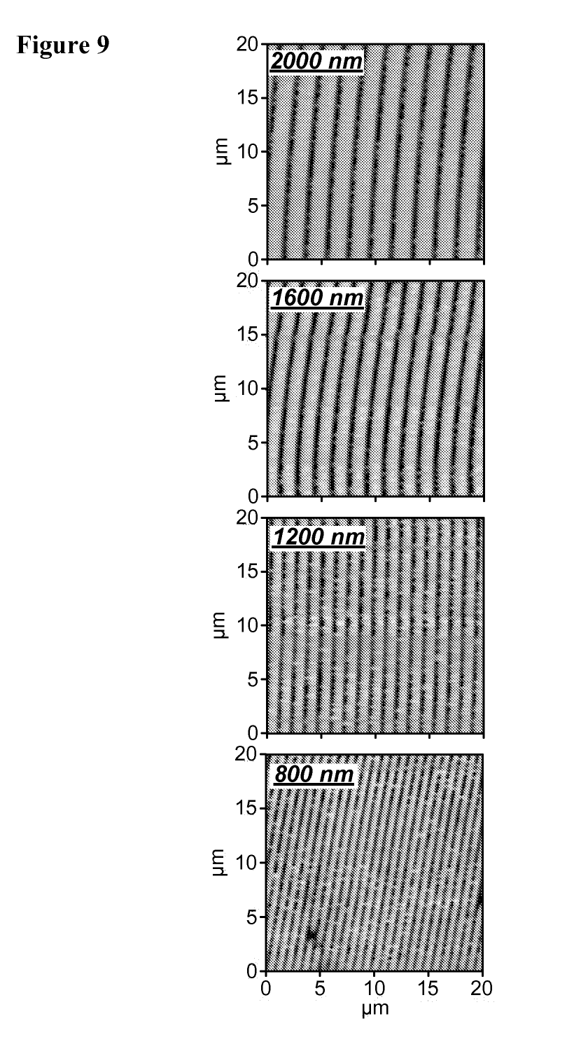

FIG. 9 shows the molded ridge-and-groove topographies in the thin film hydrogels, ranging from 2000-800 nm pitch. The elastic modulus for each of these topographies was similar to the 4000 nm pitch surface. The thin bridges observed at 800 nm are an artifact of strong AFM tip-sample adhesions. FIG. 9 shows AFM images from 2000, 1600, 1200 and 800-nm pitch ridge-and-groove patterns. 400 nm-pitch ridges and grooves were observed as doublets, indicating that the soft ridges were not capable of resisting a strong attractive force between the 200 nm spaces of the groove. For each topographic ridge and groove dimension, the elastic modulus (EM) was measured (over ridge). No trends in EM were observed as a function of pitch, and the mean elastic modulus for all the topographies was found to be 101±24 kPa. The lowest dimension to maintain the topographic features of the PDMS mold was 800 nm-pitch, i.e. 400 mn ridges and grooves. This is the smallest feature that has been molded with such a low elastic modulus.

The long-term stability of any topography is important for cell culture experiments and the substrates were found to retain their morphology, so long as only one dry-to-wet transition occurred (i.e. the first one). AFM cross sections of the 4000 nm pitch surface at 1, 2, 3 and 24 hours were compared and no changes in morphology were found. If the samples went through multiple transitions, the topography would increasingly deteriorate. The ability of the thin film hydrogels to maintain the topographic features of the mold was also highly dependent on the thickness of the thin film between the PDMS stamp and glass surface. The threshold thickness necessary for the hydrogel to maintain the topographic features when swollen was investigated by spin coating thin films of PDMS to create spacers between the mold and glass. We tested ˜18, 30 and 50 micrometer spacers and found that the hydrogel was unable to maintain the topographic features of the PDMS mold with fidelity when swollen. Also, for these thicker films, crack propagation and peeling occurred after the mold was removed. A very thin film, on the other hand, better ensures proper adhesion to the base substrate and promotes expansion of the hydrogel in a direction predominantly normal to the rigid support. Assuming that the entire 3 μL of prepolymer solution was contained under the one square centimeter of the PDMS mold, then the thin film would be 3 μm thick. However, some liquid build-up was observed around the edges of the mold, indicating the films were less than 3 μm.

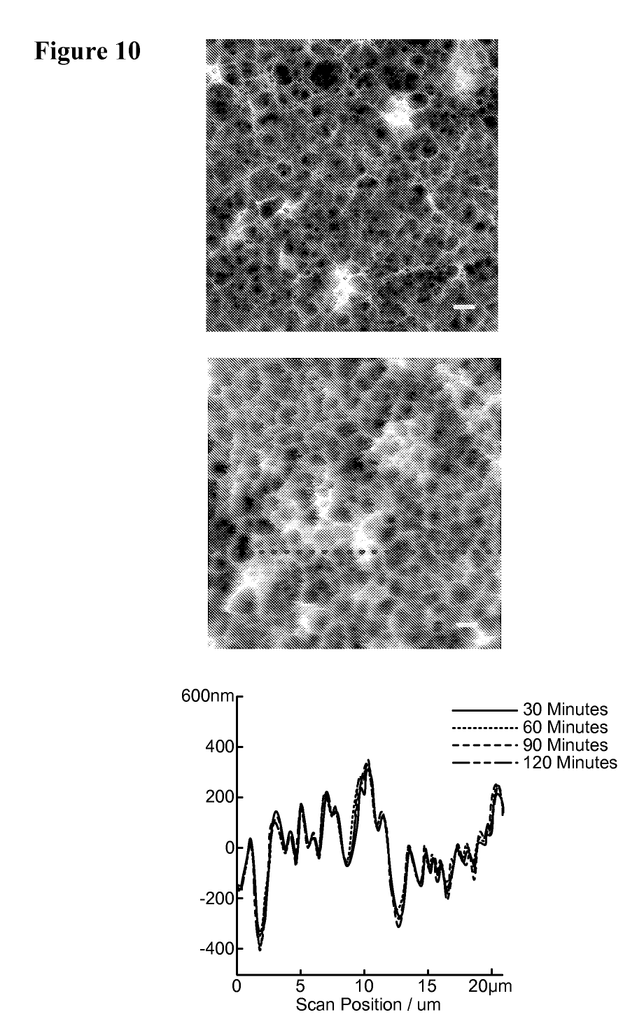

Ridge-and-groove features are commonly used to understand how topographic features influence cell behavior and therefore these results will have a significant impact on future studies. The topographic features of many basement membranes display ridges, grooves, bumps and pores with submicron dimensions, in a stochastic setting. As discussed above, the highly porous features in polyelectrolyte multilayers of PAA and PAH closely approximate in vivo basement membrane topography and serve as better membrane mimics. FIG. 10, top (AFM height image in air), depicts the in vivo-type topography cast in stiff NOA81, suitable for cell culture, as described above. The artificial topography encapsulates the sum of the topographic components of in vivo topography, e.g. fibers, bumps and pores, with stochastic surface order.

FIG. 10, middle, depicts the same topography, molded using a 6000 MW PEGDA and the thin film molding process (not same location as top panel; AFM height image in ethanol). Strong adhesions between the AFM tip and sample induce artifacts into the image of the molded topography and make it difficult to resolve the very small features of the mold. However, it is still clearly seen that a number of prominent features associated with the simulated topography are molded into the thin film hydrogel. The elastic modulus was measured over surface features with significant height components (i.e. the ridges) and was found to have a slightly higher elastic modulus value (183±39 kPa), as compared to the ridge-and-groove surfaces. This may be due to differences in the film thickness between the ridge-and-groove and synthetic membrane topography. Contributions from an underlying rigid surface are known to increase the measured elastic modulus of thin films. FIG. 10 (bottom) compares multiple scans of a cross-section of the soft topographic film as a function of time. This demonstrates the stability of the topographic features, so long as the sample remains wet and does not transition between wet and dry states.

When developing cell culture-ware for use in the laboratory, it is important to consider not only the design of the physical mimics, but also the difficulty and cost associated with replicating the surfaces in bulk. The method presented here is both cost effective and easy to implement, given the small volume of PEGDA that is necessary for the thin film. For example, from a single gram of PEGDA, one could potentially fabricate ˜1,600 individual square centimeter surfaces. One of skill in the art will appreciate that the methods of the present invention could be extended to any number of topographic features contained in a PDMS mold, in addition to the ridges and grooves and simulated basement membrane described above. Notably, submicron topographic features can be replicated with an elastic modulus that is comparable to that sensed by cells in the body.

### EXAMPLE 5

PEGDA-Laponite nanocomposite solution preparation. Irgacure 2595 was dissolved in ultrahigh purity water at 0.015% (w/v). Sonication (15-20 minutes) was used to dissolve the Irgacure. To this solution, Laponite XLS clay (synthetic hectorite) was added at concentrations that range from 0.5-2.0% (w/v). Individual Laponite particles were approximately 3 nm in diameter and hydrated and swelled in water. Dissolution and exfoliation of the Laponite was conducted using 30 minutes of mixing with a stirbar to achieve a clear solution in water. To this solution, PEGDA (MW=700) was added at concentrations ranging between 10 and 50% (w/v).

Pre-film substrate preparation. Glass surfaces were plasma treated in air for 2 minutes. These surfaces were immediately placed in a vaccuum chamber with a small volume of 3-acryloxypropyltrichlorosilane (APT). The vacuum chamber was evacuated and APT vapor silanated the surface for 1 hour, to produce a surface which contained covalently bound acrylate groups. These groups were used for crosslinking PEGDA to the surface of the glass, to produce a strongly bound PEGDA-Laponite thin film. After 1 hour in the presence of APT, the APT was removed and the glass surface was held under vacuum for 24 hours to remove any unreacted APT.

Formation of the PEGDA-Laponite nanocomposite thin film. 6 microliters of the PEGDA-Laponite solution was pipetted onto an APT-coated glass slide. A one square micrometer PDMS mold with alternating parallel ridges and grooves with a pitch of four micrometers (pitch=ridge+groove length), was pressed on top of the 6 microliter drop of the PEGDA-Laponite solution, to produce a thin film between the PDMS mold and APT coated glass slide. This assembly was placed in a plastic bag containing an atmosphere of high purity nitrogen. This was left to rest in the nitrogen gas bag for 30 minutes. The PEGDA-Laponite thin film was then UV-cured for 20 minutes in the nitrogen bag. The PDMS mold was then gently peeled from the cured PEGDA-Laponite thin film.

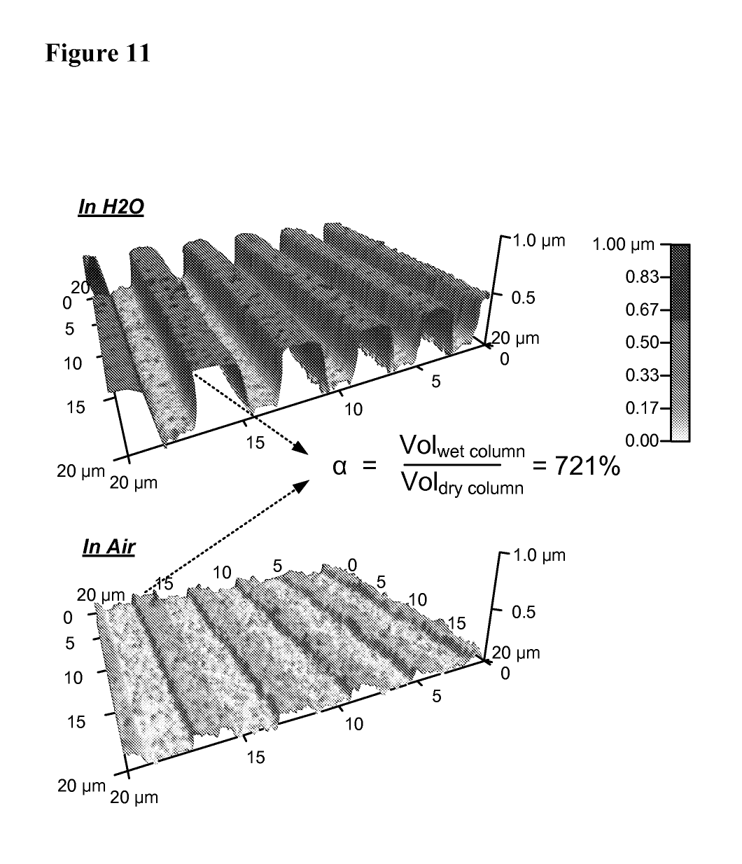

The PEGDA-Laponite thin film was imaged using atomic force microscopy. The column volume of the ridges increased by over 700% after swelling, as compared to the pattern in the dry state (FIG. 11). The novel, transparent nanocomposite thin film can be reversibly switched from a flat surface to a surface with well defined nanotropographic features by exposing the thin film to solutions containing water or ethanol. Upon drying, the surface returns to a nondescript topography (flat surface). The film can reversibly switch between a flat surface and a high fidelity nanotopography formed by soft lithography.

Although the foregoing invention has been described in some detail by way of illustration and example for purposes of clarity of understanding, one of skill in the art will appreciate that certain changes and modifications may be practiced within the scope of the appended claims. In addition, each reference provided herein is incorporated by reference in its entirety to the same extent as if each reference was individually incorporated by reference. Where a conflict exists between the instant application and a reference provided herein, the instant application shall dominate.

1. A method for preparing a nanostructured membrane, the method comprising:
a) providing a substrate with a charged silanized surface;
b) forming a multilayered membrane comprising a first polyelectrolyte and a second polyelectrolyte, wherein the membrane contacts the silanized surface;
c) inducing polyelectrolyte phase separation;
d) crosslinking the multilayered membrane; and
e) covalently linking the multilayered membrane to the silanized surface.

2. The method of claim 1, wherein the substrate is plastic or glass.

3. The method of claim 1, wherein step a) comprises contacting the substrate with (3-aminopropyl)-trimethoxysilane.

4. The method of claim 1, wherein the multilayered membrane comprises 5-25 layers of the first polyelectrolyte and an equal number of layers of the second polyelectrolyte, and wherein the layers of the first polyelectrolyte and the second polyelectrolyte contact each other in an alternating fashion.

5. The method of claim 4, wherein the multilayered membrane comprises 10-15 layers of the first polyelectrolyte and an equal number of layers of the second polyelectrolyte.

6. The method of claim 1, wherein the first polyelectrolyte is poly(acrylic acid).

7. The method of claim 1, wherein the second polyelectrolyte is poly(allylamine hydrochloride).

8. The method of claim 1, wherein step c) comprises contacting the multilayered membrane with an acidic aqueous solution.

9. The method of claim 1, wherein steps d) and e) are performed concurrently by heating the multilayered membrane and the substrate.

10. The method of claim 1, wherein steps d) and e) are performed concurrently by contacting the multilayered membrane and the substrate with a chemical crosslinker.

11. The method of claim 10, wherein the chemical crosslinker is glutaraldehyde.

12. The method of claim 1, further comprising:
f) preparing a composite stamp using the crosslinked multilayered membrane as a template;
g) imprinting an uncured polymer substrate with the stamp; and
h) curing the polymer substrate to form a membrane replica.

13. The method of claim 12, wherein step f) comprises:
i) coating the crosslinked multilayered membrane with a surfactant;
ii) forming a thin layer of stiff poly(dimethylsiloxane) (PDMS) in contact with the surfactant-coated membrane;
iii) forming a thick layer of flexible PDMS in contact with the thin layer of stiff PDMS; and
iv) removing the resulting two-layer composite stamp from the multilayered membrane.

14. The method of claim 12, wherein step g) comprises:
i) coating a surface with a pre-polymer comprising a mercapto-ester; and
ii) pressing the composite stamp into the pre-polymer to form an imprinted uncured polymer substrate.

15. The method of claim 12, wherein step g) comprises:
i) contacting a surface with (3-acryloxypropyl)-trichlorosilane; and
ii) forming an aqueous film between the surface and the composite stamp, wherein the film comprises polyethylene glycol diacrylate and a photoinitiator, to form an imprinted uncured polymer substrate.

16. The method of claim 15, wherein the aqueous film further comprises a component selected from collagen, laponite, and mixtures thereof.

17. The method of claim 14, wherein step h) comprises exposing the imprinted uncured polymer substrate to ultraviolet light.

18. A nanostructured membrane comprising:
a) a substrate with a silanized surface; and
b) a crosslinked multilayered membrane contacting the silanized surface and comprising a first polyelectrolyte and a second polyelectrolyte;
wherein the crosslinked membrane is covalently linked to the silanized surface, and wherein the crosslinked membrane comprises pores ranging from about 50 nanometers to about 1.5 micrometers.

19. A membrane replica fabricated according to the method of claim 12.

20. A cell culture substrate comprising a membrane according to claim 18.

21. A tissue culture substrate comprising a membrane according to claim 18.

22. An implantable prosthetic device comprising a membrane according to claim 18.

23. A method for preparing a compliant nanostructured surface, the method comprising:
a) contacting a surface with (3-acryloxypropyl)-trichlorosilane;
b) forming an aqueous film between the surface and a nanostructured mold, wherein the film comprises polyethylene glycol diacrylate and a photoinitiator, to form an imprinted uncured polymer substrate; and
c) exposing the imprinted uncured polymer substrate to ultraviolet light to form the compliant surface.

24. The method of claim 23, wherein the aqueous film further comprises laponite particles, and wherein the compliant nanostructured surface can be reversibly switched between a flat surface and a surface with nanotopographic features.

25. The method of claim 23, wherein the aqueous film further comprises one or more substances selected from the group consisting of collagen, fibronectin, laminin, polylysine, and an RGD peptide.

26. The method of claim 23, wherein the nanostructured mold comprises a PDMS stamp.

27. A compliant surface fabricated according to the method of claim 23.

28. A reversibly-nanostructured compliant surface fabricated according to the method of claim 24.

29. A cell culture substrate comprising a compliant surface according to claim 27.

30. A tissue culture substrate comprising a compliant surface according to claim 27.

31. An implantable prosthetic device comprising a compliant surface according to claim 27.
